(19) United States
(12) Patent Application Publication
(10) Pub. No.: US 2005/0153954 A1
(43) Pub. Date: Jul. 14, 2005

(54) Novel crystalline polymorph form-vi of olanzapine and a process for preparation thereof

(76) Inventors: Buchi Reddy Reguri, Hyderabad (IN); Ramesh Chakka, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 10/509,473

(22) PCT Filed: Apr. 22, 2003
(86) PCT No.: PCT/US03/12414

(30) Foreign Application Priority Data
Apr. 23, 2002 (IN) 311/MAS/2002

(51) Int. Cl. A61K 31/551; C07D 498/02

(52) U.S. Cl. 514/220; 540/560

(57) ABSTRACT

The present invention relates to a novel crystalline form of 2-methyl-4-4-methyl-piperazinyl)-10H-thieno[2,3-b][1,5] benzodiazepine and to a method of preparation thereof. Methyl-4-(4-methyl-1-piperazinyl)-10H-thieno[2,3-b][1,5]benzodiazepine (Olanzapine) is represented by Structure (I).

### FIELD OF INVENTION

The present invention relates to a novel crystalline form of 2-methyl-4(4-methyl-piperazinyl)-10H-thieno[2,3-b][1,5] benzodiazopine and to a method of preparation thereof. Methyl-4-(4-methyl-1-piperazinyl)-10H-thieno[2,3-b][1,5]benzodiazepine (Olanzapine) is represented by the following Structure.

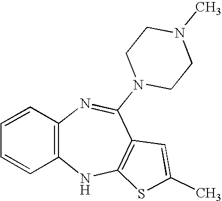

The present invention also relates to compositions made using the crystalline form of 2-methyl-4-(4-methyl-piperazinyl)-10H-thieno[2,3-b][1,5]benzodiazepine and the use of crystalline form and compositions made using the crystalline form for the treatment of disorders of the central nervous system, for treating psychotic patients and mild anxiety.

### BACKGROUND OF INVENTION

U.S. Pat. No. 5,229,382 discloses the preparation of Olanzapine and its acid addition salts, having pharmaceutical properties, particularly in the treatment of disorders of the central nervous system. The patent does not refer to any specific polymorphic crystalline forms of Olanzapine.

EP 0 733 635 A1 claims Form-2 of Olanzapine. The patent also states that the product obtained according to the process described in U.S. Pat. No. 5,229,382 as Olanzapine Form-I.

Furthermore, EP 0 733 635 A1 discloses the d values for Form-1 and Form-2 from their X-ray Diffractograms. The d values are as follows:

| d value | d value |
|---|---|
| Form-1 | Form-2 |
| 9.94 | 10.26 |
| 8.55 | 8.57 |
| 8.24 | 7.47 |
| 6.88 | 7.12 |
| 6.37 | 6.14 |
| 6.24 | 6.07 |
| 5.58 | 5.48 |
| 5.30 | 5.21 |
| 4.98 | 5.12 |
| 4.83 | 4.98 |
| 4.72 | 4.76 |
| 4.62 | 4.71 |
| 4.53 | 4.47 |
| 4.46 | 4.33 |
| 4.29 | 4.22 |
| 4.23 | 4.14 |
| 4.08 | 3.98 |
| 3.82 | 3.72 |
| 3.74 | 3.56 |
| 3.69 | 3.53 |
| 3.58 | 3.38 |
| 3.50 | 3.25 |
| 3.33 | 3.12 |
| 3.28 | 3.08 |
| 3.21 | 3.06 |
| 3.11 | 3.01 |
| 3.05 | 2.87 |
| 2.94 | 2.81 |
| 2.81 | 2.72 |
| 2.75 | 2.64 |
| 2.65 | 2.60 |
| 2.63 | |
| 2.59 | |

EP 831098B1 discloses Olanzapine Form-Il as the most stable anhydrous form of Olanzapine, providing a stable anhydrous formulation with pharmaceutically desired characteristics. The patent further discloses that substantially pure Olanzapine Form-II, which can be prepared using an Olanzapine dehydrate. In addition to this, the patent discloses the preparation of a series of dihydrates of Olanzapine namely Dehydrate B, Dehydrate D and Dehydrate E characterized by their XRD pattern which serve as intermediates for the preparation of Olanzapine Form-II.

U.S. Pat. No. 6,348,458 B1 discloses the preparation of a series of crystalline polymorphic forms of Olanzapine namely Form-III, Form-IV and Form-V. The d values for these forms from their X-Ray Diffractograms are also incorporated in the patent and are mentioned in the following Table-1.

| Form-III | Form-IV | Form-V |
|---|---|---|
| d value | d value | d value |
| 10.7476 | 9.9487 | 10.5932 |
| 10.3156 | 8.5074 | 10.217 |
| 8.6245 | 8.2103 | 9.9503 |
| 7.1713 | 6.8673 | 8.5259 |
| 6.5014 | 4.9734 | 7.1016 |
| 6.112 | 4.8172 | 6.0731 |
| 5.9251 | 4.7114 | 5.2041 |
| 5.8243 | 4.6122 | 4.9856 |
| 5.5165 | 4.5282 | 4.8153 |
| 5.2359 | 4.234 | 4.7514 |
| 4.8541 | 4.0901 | 4.6139 |
| 4.7514 | 3.7574 | 4.5302 |
| 4.5578 | 3.6989 | 4.4714 |
| 4.4938 | 3.5052 | 4.2271 |
| 4.4536 | | 4.1307 |
| 4.2588 | | 4.0736 |
| 4.1523 | | 3.988 |
| 4.0699 | | 3.7763 |
| 3.9898 | | 3.7167 |
| 3.8955 | | 3.5315 |
| 3.7288 | | 3.3762 |
| 3.5626 | | 3.006 |
| 3.0262 | | |

WO 02/18390 discloses Olanzapine monohydrate I and Olanzapine dehydrate I and process for making these compounds.

The novel crystalline polymorphic form of Olanzapine of the present invention is well distinguished from the crystalline polymorphic forms reported in the prior art and conveniently herein after, designated as Polymorph Form-VI of Olanzapine. Hence present invention provides a novel crystalline polymorph Form-VI of Olanzapine and the present invention also embodies the process for the preparation of crystalline polymorph Form-VI of Olanzapine, more specifically the present invention is related to conversion of Polymorph Form-I of Olanzapine to novel crystalline polymorph Form-VI of Olanzapine.

### SUMMARY OF THE INVENTION

The present invention provides a novel crystalline polymorphic Form of 2-methyl-4-(4-methyl-1-piperazinyl)-10H-thieno[2,3-b][1,5]benzodiazepine (Olanzapine), conveniently designated as Polymorph Form-VI of Olanzapine. The invention also relates to provide the process for the preparation of crystalline polymorph Form-VI of Olanzapine, which comprises stirring of polymorph Form-I of Olanzapine in a C₁-C₆ alkanol to obtain the novel crystalline polymorph Form-VI of Olanzapine.

The process of the present invention is eco friendly and well suited for industrial scale up.

### BRIEF DESCRIPTION OF ACCOMPANYING DRAWINGS

FIG. 1 is an X-ray powder diffractogram of the Form VI obtained in the present invention.

FIG. 2 is a DSC thermogram of the Form VI obtained in the present invention.

FIG. 3 is an infrared absorption spectrum of the Form VI obtained in the present invention.

### DETAILED DESCRIPTION OF THE INVENTION

Accordingly, the present invention provides novel crystalline polymorph Form-VI of Olanzapine and a process for the preparation thereof.

The Crystalline nature of polymorph Form-VI of Olanzapine can be characterized by its X-ray diffractogram, Infrared spectrum and Differential scanning calorimetry thermogram.

The X-ray powder diffraction pattern of crystalline polymorph Form-VI of Olanzapine was measured on a Rigaku D/Max 2200 Powder Diffractometer with Cu Radiation source. The Crystalline polymorph Form-VI of Olanzapine has X-ray powder diffraction pattern essentially as shown in the Table-2. The X-ray powder diffraction pattern is expressed in the terms of its d values, and percentage intensity (in %).

TABLE 2

| | Intensity |
|---|---|
| d-values | (%) |
| 10.2972 | 35 |
| 8.5646 | 6 |
| 7.6618 | 22 |
| 7.4935 | 21 |
| 7.3691 | 21 |
| 6.6317 | 25 |
| 6.5246 | 29 |
| 6.2320 | 87 |
| 5.7713 | 7 |
| 5.7121 | 9 |
| 5.3042 | 20 |
| 5.2174 | 6 |
| 4.9733 | 34 |
| 4.8335 | 7 |
| 4.7614 | 5 |
| 4.7162 | 8 |
| 4.6284 | 27 |
| 4.4802 | 13 |
| 4.3795 | 54 |
| 4.3163 | 77 |
| 4.2874 | 100 |
| 4.2308 | 21 |
| 4.1297 | 34 |
| 4.0958 | 34 |
| 4.0117 | 17 |
| 3.8275 | 24 |
| 3.7263 | 13 |
| 3.6509 | 17 |
| 3.5311 | 6 |
| 3.3141 | 29 |
| 3.2782 | 18 |
| 3.1207 | 17 |
| 3.0035 | 5 |
| 2.8824 | 5 |
| 2.8099 | 8 |
| 2.8014 | 6 |
| 2.0562 | 6 |

The present invention of crystalline polymorph Form-VI of Olanzapine is characterized by its X-ray powder diffraction as depicted in FIG. (1).

The present invention also provides Differential Scanning Calorimetry thermogram of crystalline polymorph Form-VI of Olanzapine. The Differential Scanning Calorimetry thermogram exhibits a significant endo peak around 196° C. and as depicted in FIG. (2).

The present invention further provides the Infrared data for crystalline polymorph Form-VI of Olanzapine, which was measured by KBr-transmission method with identified significant peaks around 3217 cm⁻¹, 2933 cm⁻¹, 1592 cm⁻¹, 1561 cm⁻¹, 1468 cm⁻¹, 1369 cm⁻¹, 1218 cm⁻¹, 1143 cm⁻¹, 1007 cm⁻¹, 964 cm⁻¹ , 751 cm⁻¹ and 674 cm⁻¹. The present invention provides the IR spectrum of crystalline polymorph Form-VI of Olanzapine as depicted in FIG. (3).

Accordingly, the present invention provides novel crystalline polymorphic Form-VI of 2-methyl-4-(4-methyl-1-piperazinyl)-10H-thieno[2,3-b][1,5]benzodiazepine (Olanzapine), which comprises:
i) stirring polymorph Form-I of Olanzapine in a C₁-C₆ alkanol at a temperature of 0 to 40° C. for 30 minutes to 10 hours;
ii) isolating the obtained solid form step (i) by conventional methods;
iii) drying the compound of step (ii) at a temperature of 40 to 100° C. to afford the desired crystalline polymorph Form-VI of Olanzapine.

Preferably, the alkanol is n-butanol or tert.butanol. More preferably the alkanol is n-butanol.

Preferably, the solid obtained in step 2) is isolated by filtering, decanting or centrifuging.

The present invention therefore provides novel Olanzapine Form-VI and a simple method for its preparation.

The polymorph Form-I of Olanzapine was prepared as per the procedure disclosed in WO 02/18390 A1 the disclosure of which is incorporated by reference. A mixture of 4-amino-2-methyl-10H-thieno-[2,3-b][1,5]benzodiazepine HCl (100 g), N-methyl piperizine (350 ml), DMSO (465 ml) and toluene (465 ml) was heated to reflux. The reaction mass was maintained at reflux for 19 hours and then cooled to 50° C. and water was added. The reaction mass was cooled to 0-10° C. and stirred at the same temperature for 6 hours. The crude Olanzapine separated was filtered and dried in oven to a constant weight (76.5 g). The crude compound was added to acetonitrile (750 ml) at boiling temperature. The mixture was boiled for further 5 minutes. The mixture was filtered to remove the undissolved solid. The filtrate was treated with carbon and filtered. The filtrate was distilled to a minimum volume, cooled to 0-5° C. and maintained at the same temperature for 1.0 hour and filtered. The compound was dried to a constant weight in an oven (51.6 g).

Form I can be prepared by any other method.

The invention likewise relates to the use of novel crystalline of 2-methyl-4-(4-methyl-piperazinyl)-10H-thieno[2,3-b][1,5]benzodiazepine useful in the treatment of disorders of the central nervous system, for treating psychotic patients or mild anxiety. It can be used to prepare pharmaceutically acceptable preparations, in a method for the prophylactic and/or therapeutic treatment of an animal or human.

The invention likewise relates to pharmaceutical preparations made using the novel crystalline of 2-methyl-4-(4-methyl-piperazinyl)-10H-thieno[2,3-b][1,5]benzodiazepine and to processes for their preparation.

The pharmaceutical preparations according to the invention which contain the compound according to the invention are those for enteral, such as oral, furthermore rectal, and parenteral administration to (a) warm-blooded animal(s), the pharmacological active ingredient being present on its own or together with a pharmaceutically acceptable carrier. The daily dose of the active ingredient depends on the age and the individual condition and also on the manner of administration.

The novel pharmaceutical preparations contain, for example, from about 10% to about 80%, preferably from about 20% to about 60%, of the 2-methyl-4-(4 methyl-piperazinyl)-10H-thieno[2,3-b][1,5]benzodiazepine. Preferably, the compositions are formulated in unit dosage form, each dosage containing from 0.1 mg to 20 mg or 0.5 to 10 mg of the 2-methyl-4-(4-methyl-piperazinyl)-10H-thieno[2,3-b][1,5]benzodiazepine. Pharmaceutical compositions of this invention can contain and/or comprise a therapeutically effective amount of the 2-methyl-4-(4-methyl-piperazinyl)-10H-thieno[2,3-b][1,5]benzodiazepine made from the novel crystalline form of this invention, together with one or more inorganic or organic, solid or liquid, pharmaceutically acceptable carriers, diluents, excipients, additives, fillers, lubricants, binders, stabilizers, solvents or solvates. The compositions may be in the form of a tablet, capsule, lozenge, powder, syrup, solution, suspension, ointment or dragee. The pharmaceutical compositions may be sterilized and/or may comprise of one or more excipients, for example preservatives, stabilizers, wetting agents and/or emulsifiers, solubilizers, salts for regulating the osmotic pressure and/or buffers. Pharmaceutical preparations according to the invention for enteral or parenteral administration are, for example, those in unit dose forms, such as sugar-coated tablets, tablets, capsules or suppositories, and furthermore ampoules. These are prepared in a manner known per se, for example by means of conventional mixing, granulating, sugar-coating, dissolving or lyophilizing processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

Suitable carriers are, in particular, fillers, such as sugars, for example lactose, sucrose, mannitol or sorbitol, cellulose preparations and/or calcium phosphates, for example tricalcium phosphate or calcium hydrogen phosphate, furthermore binders, such as starch paste, using, for example, corn, wheat, rice or potato starch, gelatin, tragacanth, methylcellulose and/or polyvinylpyrrolidone, if desired, disintegrants, such as the above mentioned starches, furthermore carboxymethyl starch, crosslinked polyvinylpyrrolidone, agar, alginic acid or a salt thereof, such as sodium alginate; auxiliaries are primarily glidants, flow-regulators and lubricants, for example silicic acid, talc, stearic acid or salts thereof, such as magnesium or calcium stearate, and/or polyethylene glycol. Sugar-coated tablet cores are provided with suitable coatings which, if desired, are resistant to gastric juice, using, inter alia, concentrated sugar solutions which, if desired, contain gum arabic, talc, polyvinylpyrrolidone, polyethylene glycol and/or titanium dioxide, coating solutions in suitable organic solvents or solvent mixtures or, for the preparation of gastric juice-resistant coatings, solutions of suitable cellulose preparations, such as acetylcellulose phthalate or hydroxypropylmethylcellulose phthalate. Colorants or pigments, for example to identify or to indicate different doses of active ingredient, may be added to the tablets or sugar-coated tablet coatings.

Other orally utilizable pharmaceutical preparations are hard gelatin capsules, and also soft closed capsules made of gelatin and a plasticizer, such as glycerol or sorbitol. The hard gelatin capsules may contain the active ingredient in the form of granules, for example in a mixture with fillers, such as lactose, binders, such as starches, and/or lubricants, such as talc or magnesium stearate, and, if desired, stabilizers. In soft capsules, the active ingredient is preferably dissolved or suspended in suitable liquids, such as fatty oils, paraffin oil or liquid polyethylene glycols, it also being possible to add stabilizers.

Suitable rectally utilizable pharmaceutical preparations are, for example, suppositories, which consist of a combination of the active ingredient with a suppository base. Suitable suppository bases are, for example, natural or synthetic triglycerides, paraffin hydrocarbons, polyethylene glycols or higher alkanols. Furthermore, gelatin rectal capsules which contain a combination of the active ingredient with a base substance may also be used. Suitable base substances are, for example, liquid triglycerides, polyethylene glycols or paraffin hydrocarbons.

Suitable preparations for parenteral administration are primarily aqueous solutions of an active ingredient in water-soluble form, for example a water-soluble salt, and furthermore suspensions of the active ingredient, such as appropriate oily injection suspensions, using suitable lipophilic solvents or vehicles, such as fatty oils, for example sesame oil, or synthetic fatty acid esters, for example ethyl oleate or triglycerides, or aqueous injection suspensions which contain viscosity-increasing substances, for example sodium carboxymethylcellulose, sorbitol and/or dextran, and, if necessary, also stabilizers.

The dose of the active ingredient depends on the warm-blooded animal species, the age and the individual condition and on the manner of administration. In the normal case, an approximate daily dose of about 10 mg to about 350 mg is to be estimated in the case of oral administration for a patient weighing approximately 75 kg. For other types of administration, the preferred daily dose is between 0.1 mg to 1000 mg per kilogram, preferably between 1 mg to 100 mg daily for warm-blooded species. More, preferably, between 1 mg to 40 mg daily.

### DETAILED DESCRIPTION OF ACCOMPANYING DRAWINGS

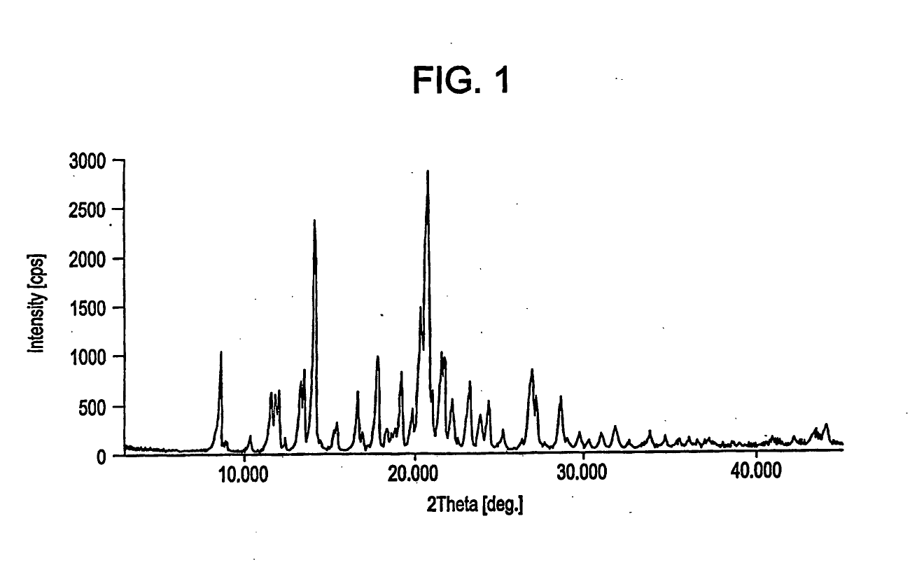

FIG. 1 is a characteristic X-ray powder diffraction pattern of novel crystalline-polymorph Form-VI of Olanzapine.

(Vertical axis: Intensity (CPS); Horizontal axis: 20(degrees).

The significant d values obtained are 10.2972, 8.5646, 7.6618, 7.4935, 7.3691 6.6317, 6.5246, 6.2320, 5.7713, 5.7121, 5.3042, 5.2174, 4.9733, 4.8335, 4.7614, 4.7162, 4.6284, 4.4802, 4.3795, 4.3163, 4.2874, 4.2308, 4.1297, 4.0958, 4.0117, 3.8275, 3.7263, 3.6509, 3.5311, 3.3141, 3.2782, 3.1207, 3.0035, 2.8824, 2.8099, 2.8014 and 2.0562 A°.

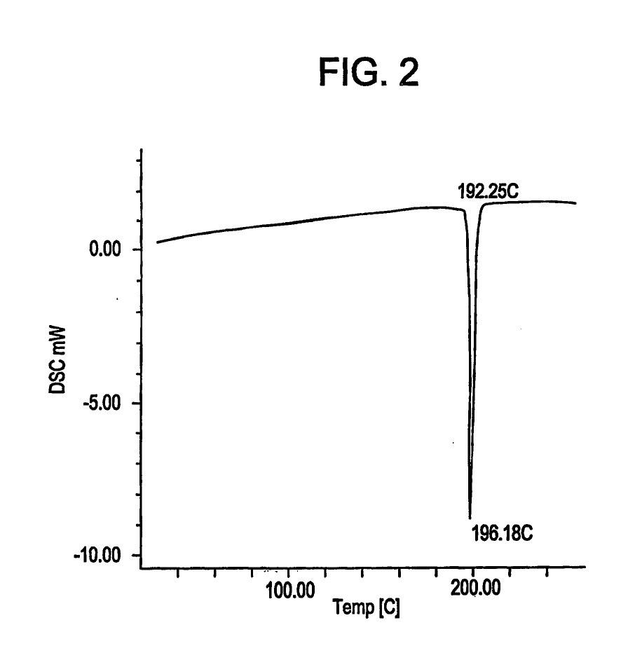

FIG. 2 is a characteristic Differential Scanning Calorimetric thermogram of novel crystalline polymorph of Form-VI of Olanzapine.

Vertical axis: Temperature (in ° C.); Horizontal axis: Signal (in mV).

The Differential Scanning Calorimetric Thermogram exhibits a significant endo peak at 196° C.

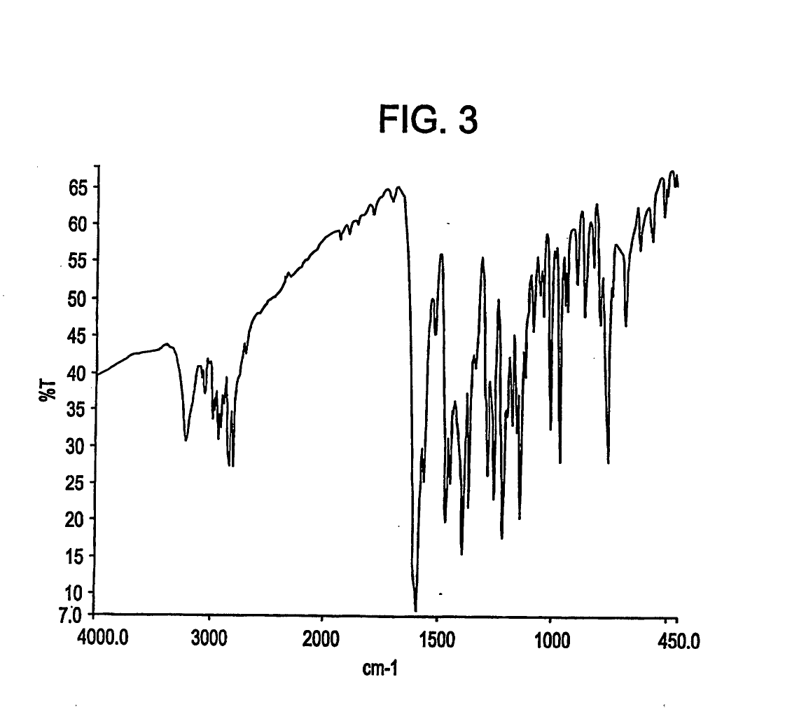

FIG. 3 is a characteristic infrared absorption spectrum in potassium bromide of Olanzapine Form-VI.

[Vertical axis, Transmission (%); Horizontal axis: Wave number (cm⁻¹)].

The characteristic peaks for Olanzapine Form-VI are indicated around 3217 cm⁻¹, 2933 cm⁻¹, 1592 cm⁻¹, 1561 cm⁻¹, 1468 cm⁻¹, 1369 cm⁻¹, 1218 cm⁻¹, 1143 cm⁻¹, 1007 cm⁻¹, 964 cm⁻¹, 751 cm⁻¹ and 674 cm⁻¹.

### EXAMPLES

The present invention is described in detail with example given below that are provided by way of illustration only and therefore, should not be construed to limit the scope of the invention.

### Preparation of Crystalline Polymorph Form-VI Of Olanzapine

### EXAMPLE 1

A mixture of polymorph Form-I of Olanzapine (10.0 g) and n-butanol (30 ml) was stirred at a temperature of 25-30° C. for 1-2 hours. Further the compound was filtered, washed with n-butanol (5.0 ml) and dried at a temperature of 60-70° C. to a constant weight to render the desired crystalline polymorph Form-VI of Olanzapine.

(Yield: 7.1 grams, 71.0%).

1. A novel crystalline polymorph Form-VI of 2-methyl-4-(4-methyl-1-piperazinyl)-10H-thieno[2,3-b][1,5]benzodiazepine (Olanzapine).

2. The crystalline polymorph Form-VI of Olanzapine of claim 1 having X-ray powder diffraction pattern with characteristic d-values (in A°) percentage (in %) as shown in the following table.

| | Intensity |
|---|---|
| d-values | (%) |
| 10.2972 | 35 |
| 8.5646 | 6 |
| 7.6618 | 22 |
| 7.4935 | 21 |
| 7.3691 | 21 |
| 6.6317 | 25 |
| 6.5246 | 29 |
| 6.2320 | 87 |
| 5.7713 | 7 |
| 5.7121 | 9 |
| 5.3042 | 20 |
| 5.2174 | 6 |
| 4.9733 | 34 |
| 4.8335 | 7 |
| 4.7614 | 5 |
| 4.7162 | 8 |
| 4.6284 | 27 |
| 4.4802 | 13 |
| 4.3795 | 54 |
| 4.3163 | 77 |
| 4.2874 | 100 |
| 4.2308 | 21 |
| 4.1297 | 34 |
| 4.0958 | 34 |
| 4.0117 | 17 |
| 3.8275 | 24 |
| 3.7263 | 13 |
| 3.6509 | 17 |
| 3.5311 | 6 |
| 3.3141 | 29 |
| 3.2782 | 18 |
| 3.1207 | 17 |
| 3.0035 | 5 |
| 2.8824 | 5 |
| 2.8099 | 8 |
| 2.8014 | 6 |
| 2.0562 | 6 |

3. The crystalline polymorph Form-VI of Olanzapine of claim 2, having an X-ray powder diffraction pattern as depicted in FIG. (1).

4. The crystalline polymorph Form-VI of Olanzapine of claim 1, having differential scanning calorimetry thermogram which exhibits a characteristic endo peak around 196° C.

5. The crystalline polymorph Form-1 of Olanzapine of claim 4, having a differential scanning calorimetry thermogram as depicted in FIG. (2).

6. The crystalline polymorph Form-VI of Olanzapine of claim 1, having identified characteristic peaks around 3217 cm⁻¹, 2933 cm⁻¹, 1592 cm⁻¹, 1561 cm⁻¹, 1468 cm⁻¹, 1369 cm⁻¹, 1218 cm−1, 1143 cm⁻¹, 1007 cm⁻¹, 964 cm⁻¹, 751 cm⁻¹ and 674 cm⁻¹ in the Infra red Spectrum.

7. The crystalline polymorph Form-VI of Olanzapine of claim 6, having an Infrared spectrum as depicted in FIG. (3).

8. A process for the preparation of novel crystalline polymorph Form-VI of 2-methyl-4-(4-methyl-1-piperazinyl)-10H-thieno[2,3-b][1,5]benzodiazepine (Olanzapine), which comprises;
(i) stirring polymorph Form-I of Olanzapine in a C₁-C₆ alkanol at a temperature of 0 to 40° C. for 30 minutes to 10 hours;
(ii) isolating the obtained solid form step (i) by conventional methods; and
(iii) drying the compound of step (ii) at a temperature of 40 to 100° C. to afford the desired crystalline polymorph Form-VI of Olanzapine.

9. The process as claimed in claim 8, of step (i), wherein the said alcohol is n-butanol or tert-butanol.

10. A composition comprising novel crystalline Form VI of 2-methyl-4-(4-methyl-1-piperazinyl)-10H-thieno[2,3-b][1,5]benzodiazepine according to any one of claims 1 to 7 and pharmaceutically acceptable carrier, diluent, excipient, additive, filler, lubricant, binder, stabilizer, solvent or solvate.

11. The composition according to claim 10, in the form of a tablet, capsule, lozenge, powder, syrup, solution, suspension, ointment, or dragee.

12. The composition according to any one of claims 10 or 11, for the treatment of a disorder of the central nervous system.

13. A method for treating a disorder of the central nervous system comprising administering an effective amount of crystalline Form VI of 2-methyl-4-(4-methyl-1-piperazinyl)-10H-thieno[2,3-b][1,5]benzodiazepine according to any one of claims 1-7 and a pharmaceutically acceptable carrier, diluent, excipient, additive, filler, lubricant, binder, stabilizer, solvent or solvate to a patient in need thereof.

14. A medicine for the treatment of a disorder of the central nervous system comprising an effective amount of crystalline Form VI of 2-methyl-4-(4-methyl-1-piperazinyl)-10H-thieno[2,3-b][1,5]benzodiazepine according to any one of claims 1-7.

15. Use of crystalline Form VI of 2-methyl-4-(4-methyl-1-piperazinyl)-10H-thieno [2,3-b][1,5]benzodiazepine according to any one of claims 1-7 for the preparation of a medicament for the treatment of a disorder of the central nervous system.
